(19) United States
(12) Patent Application Publication
(10) Pub. No.: US 2014/0327875 A1
(43) Pub. Date: Nov. 6, 2014

(54) ADVANCED ELECTRO-ACTIVE OPTIC DEVICE

(71) Applicants: Ronald Blum, Roanoke, VA (US); William Kokonaski, Gig Harbor, WA (US)

(72) Inventors: Ronald Blum, Roanoke, VA (US); William Kokonaski, Gig Harbor, WA (US)

(21) Appl. No.: 14/003,781

(22) PCT Filed: Mar. 8, 2012
(86) PCT No.: PCT/US12/28354

§ 371 Date: Apr. 4, 2014

Related U.S. Application Data
(60) Provisional application No. 61/450,149, filed on Mar. 8, 2011.

(51) Int. Cl. G02C 7/02; A61F 2/16; G02C 7/04

(52) U.S. Cl. CPC G02C 7/022; G02C 7/049; A61F 2/1624; A61F 2/1648
USPC 351/159.03; 351/159.4; 623/6.22

(57) ABSTRACT

Ophthalmic lenses are described including an ophthalmic base, and a plurality of electro-active elements, such as dynamic micro-lenses or micro-prismatic apertures. Each electro-active element may be configured to dynamically change optical power. The ophthalmic lens may be configured such that an optical power of the ophthalmic lens focuses mostly one image at one time on the retina of the eye of the wearer. The ophthalmic lens may be, for example, a spectacle lens, other types of specialty lenses such as used for gaming and the like, a contact lens, an intra-ocular lens, and intra-ocular optic, etc. Each electro-active element may include liquid crystal, such as dichroic, non-dichroic, nematic, and/or cholesteric liquid crystal. The electro-active elements may comprise non-dichroic liquid crystal, and gaps between the electro-active elements may include a dichroic liquid crystal, or the electro-active elements may be shaped and arranged in a substantially conformal pattern.

### CROSS-REFERENCES TO RELATED APPLICATIONS

This application claims the benefit of U.S. Ser. No. 61/450,149 filed on Mar. 8, 2011, the contents of which is incorporated by reference in its entirety.

### BACKGROUND OF THE INVENTION

The present invention relates to ophthalmic lenses, which may include, for example, spectacle lenses, contact lenses, intraocular optics, intraocular lenses, etc. More specifically, the present invention relates to ophthalmic lenses including a plurality of dynamic micro-lenses or dynamic micro-prismatic apertures.

There are two major conditions that affect an individual's ability to focus on near and intermediate distance objects: presbyopia and aphakia. Presbyopia is the loss of accommodation of the crystalline lens of the human eye that often accompanies aging. In a presbyopic individual, this loss of accommodation first results in an inability to focus on near distance objects and later results in an inability to focus on intermediate distance objects. It is estimated that there are approximately 90 million to 100 million presbyopes in the United States. Worldwide, it is estimated that there are approximately 1.6 billion presbyopes.

FIG. 1 shows a cross section of a healthy human eye 100. The white portion of the eye is known as the sclera 110. The sclera is covered with a clear membrane known as the conjunctiva 120. The central, transparent portion of the eye that provides most of the eye's optical power is the cornea 130. The iris 140, which is the pigmented portion of the eye and forms the pupil 150. The sphincter muscles constrict the pupil and the dilator muscles dilate the pupil. The pupil is the natural aperture of the eye. The anterior chamber 160 is the fluid-filled space between the iris and the innermost surface of the cornea. The crystalline lens 170 is held in the lens capsule 175 and provides the remainder of the eye's optical power. A healthy lens is capable of changing its optical power such that the eye is capable of focusing at far, intermediate, and near distances, a process known as accommodation. The posterior chamber 180 is the space between the back surface of the iris and the front surface of the retina 190. The retina is the “image plane” of the eye and is connected to the optic nerve 195 which conveys visual information to the brain.

The standard tools for correcting presbyopia are reading glasses, multifocal ophthalmic lenses, and monocular fit contact lenses. Reading glasses have a single optical power for correcting near distance focusing problems. A multifocal lens is a lens that has more than one focal length (i.e., optical power) for correcting focusing problems across a range of distances. Multifocal lenses are used in eyeglasses, contact lenses, corneal inlays, corneal onlays, and intraocular lenses (IOLs). Multifocal ophthalmic lenses work by means of a division of the lens's area into regions of different optical powers. Multifocal lenses may be comprised of continuous surfaces that create continuous optical power as in a Progressive Addition Lens (PAL). Alternatively, multifocal lenses may be comprised of discontinuous surfaces that create discontinuous optical power as in bifocals or trifocals. Monocular fit contact lenses are two contact lenses having different optical powers. One contact lens is for correcting mostly far distance focusing problems and the other contact lens is for correcting mostly near distance focusing problems.

Electronic ophthalmic lenses for presbyopic wearers (those over the age of 40 years who have difficulty seeing clearly at near distances of 14-18 inches and/or intermediate distances of 18+ inches to 36 inches) have been taught for contact lenses, intra ocular lenses and spectacle lenses. Movement of the contact lens on the wearer's cornea presents a tremendous optical correction challenge following the blink of the wearer's eye having the contact lens, and with intra ocular lenses (IOLs) precise alignment of the IOL with the line of sight of the eye is critical and often missed. Thus for both electronic contact lenses and IOLs alignment and proper centration of these ophthalmic lenses is critical to the quality of vision of the wearer/user.

Alternate approaches are also being used to correct presbyopia. One approach is a corneal inlay that provides a small, fixed diameter aperture. By way of example only, the ACI 7000 corneal inlay made by AcuFocus is approximately 3.8 mm in diameter, 10 μm thick, and contains an opaque annulus with a 1.6 mm diameter transparent opening. This opening acts to reduce the aperture of the human eye to a smaller diameter than what is normally achievable by the natural constriction of the pupil.

The AcuFocus corneal inlay is designed to reduce the amount of light which reaches the retina. Additionally, the inlay is usually only be implanted in one eye as deleterious optical effects such as halos, doubling of vision, light scattering, glare, loss of contrast sensitivity, and/or reduction of light hitting the retina are too great and may be unacceptable when the inlay is implanted in both eyes. These deleterious effects are caused by the size of the inlay's aperture and occluded annulus in relation to the size of the pupil. These effects especially occur at night when the pupil dilates.

Another approach for correcting presbyopia is corneal refractive surgery in which one eye is corrected for far distance and the other eye is corrected for near distance. Another approach is a corneal inlay that provides a multifocal effect using diffractive optics, for example.

However, each of these approaches for correcting presbyopia has drawbacks. Of course, some of these drawbacks are more severe than others. For example, while spectacle eyewear is capable of correcting one's vision for far, near and intermediate distances, this approach requires wearing a device that takes away from one's natural appearance.

Approaches for correcting presbyopia that include the use of contact lenses can cause discomfort and can also result in one or more of: halos, doubling of vision, light scattering, glare, loss of contrast sensitivity, limited range of focus, and/or reduction of light hitting the retina. Approaches that include the use of IOLs can result in one or more of: light scattering, glare, halos, ghosting, loss of contrast sensitivity, limited range of focus, and/or reduction of light hitting the retina.

With regard to electronic spectacle lenses there is a need for improved and novel ways to create increased dynamic optical power while not increasing dispersion and/or light scatter. Presently with either static or dynamic diffractive optics, the larger the diffractive optic and/or the higher the optical power there is an increase in the amount of dispersion, decrease in the diffractive efficiency, and for all practical purposes a decrease in the usable portion of the diffractive optic which allows for clear vision for the user/wearer.

### BRIEF SUMMARY OF THE INVENTION

According to first aspects of the invention, an ophthalmic lens may be provided comprising an ophthalmic base, and a plurality of dynamic micro-lenses. Each micro-lens may be configured to dynamically change optical power. In embodiments, the ophthalmic lens may be configured such that an optical power of the ophthalmic lens focuses mostly one image at one time on the retina of the eye of the wearer. The ophthalmic lens may be, for example, a spectacle lens, other types of specialty lenses such as used for gaming and the like, a contact lens, an intra-ocular lens, and intra-ocular optic, etc.

In embodiments, the ophthalmic lens may be an electro-active lens. In embodiments, each micro-lens may be electronically activated. In embodiments, each micro-lens may comprise liquid crystal. In embodiments, the liquid crystal may be dichroic, or non-dichroic. In embodiments, the liquid crystal may be nematic, or cholesteric.

In embodiments, each micro-lens may comprise non-dichroic liquid crystal, and gaps between the micro-lenses may include a dichroic liquid crystal.

In embodiments, the optical power of the ophthalmic lens may focus mostly one image at one time on the retina of the eye of the wearer by reducing an amount of light passing through a portion of the lens, such as the dichroic liquid crystal.

In embodiments, the optical power of the ophthalmic lens may focus mostly one image at one time on the retina of the eye of the wearer by virtue of a fill factor of an area covered by the plurality of micro-lenses.

In embodiments, the ophthalmic lens may include a gradient of dynamic optical power.

In embodiments, the lens, such as a contact lens, may be configured to switch optical power based on a lid blink, or other cues.

In embodiments, the dynamic micro-lenses may be diffractive, or refractive.

In embodiments, the dynamic micro-lenses may include, for example, a surface relief diffractive structure, a pixilated structure, or a Fresnel structure.

In embodiments, the diameter of the micro-lens may be in a range of approximately 0.50 mm and 2.00 mm, or 1.0 mm and 1.60 mm. In embodiments, an optical power of the electro-active lens, when activated, may be in a range of approximately +1.00 D and +4.00 D, or approximately +1.00 D and +2.50 D.

In embodiments, the plurality of micro-lenses may be shaped and arranged within the ophthalmic base in substantially conformal pattern. In embodiments, an outer shape of each micro-lens may be substantially hexagonal. In embodiments, the plurality of micro-lenses may be arranged within the ophthalmic base in a honeycomb pattern.

In embodiments, a shape of each micro-lens may be substantially round.

In embodiments, the plurality of micro-lenses may be arranged within the ophthalmic base in a pattern of rings around a single micro-lens.

According to further aspects of the invention, an ophthalmic lens may comprise an ophthalmic base, and a plurality of micro-prismatic apertures. Each micro-prismatic aperture may be configured to dynamically change prismatic power. In embodiments, the micro prismatic apertures may be configured such that a prismatic power of the ophthalmic lens focuses mostly one image at one time on the retina of the eye of the wearer. The ophthalmic lens may be, for example, a spectacle lens, other types of specialty lenses such as used for gaming and the like, a contact lens, an intra-ocular lens, and intra-ocular optic, etc.

In embodiments, the ophthalmic lens may be an electro-active lens. In embodiments, each micro-prismatic aperture may be electronically activated. In embodiments, each micro-prismatic aperture may comprise liquid crystal. In embodiments, the liquid crystal may be dichroic, or non-dichroic. In embodiments, the liquid crystal may be nematic, or cholesteric.

In embodiments, each micro-prismatic aperture may comprise non-dichroic liquid crystal, and gaps between the micro-prismatic aperture may include a dichroic liquid crystal.

In embodiments, the optical power of the ophthalmic lens may focus mostly one image at one time on the retina of the eye of the wearer by reducing an amount of light passing through a portion of the lens, such as the dichroic liquid crystal.

In embodiments, the optical power of the ophthalmic lens may focus mostly one image at one time on the retina of the eye of the wearer by virtue of a fill factor of an area covered by the plurality of micro-prismatic aperture.

In embodiments, the ophthalmic lens may include a gradient of dynamic optical power.

In embodiments, the diameter of the micro-prismatic aperture may be in a range of approximately 0.50 mm and 2.00 mm, or 1.0 mm and 1.60 mm.

In embodiments, the plurality of micro-prismatic apertures may be shaped and arranged within the ophthalmic base in substantially conformal pattern. In embodiments, an outer shape of each micro-prismatic aperture may be substantially hexagonal. In embodiments, the plurality of micro-prismatic apertures may be arranged within the ophthalmic base in a honeycomb pattern.

In embodiments, a shape of each micro-prismatic aperture may be substantially round.

In embodiments, the plurality of micro-prismatic apertures may be arranged within the ophthalmic base in a pattern of rings around a single micro-prismatic aperture.

### BRIEF DESCRIPTION OF THE DRAWINGS

Aspects and features of the invention will be understood and appreciated more fully from the following detailed description in conjunction with the figures, which are not to scale, in which like reference numerals indicate corresponding, analogous or similar elements.

FIG. 1 shows a cross section of a human eye.

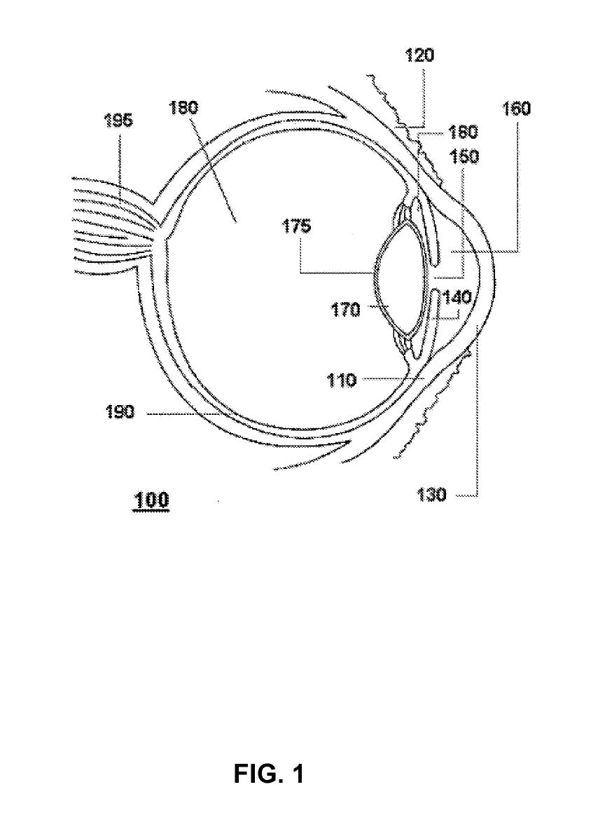

FIG. 2 shows a first embodiment of a lens including a plurality of electro-active elements with diffractive regions, surrounded by a dichroic crystal region, according to aspects of the invention.

FIG. 3 shows another embodiment of a lens including a plurality of electro-active elements with diffractive regions according to aspects of the invention.

FIG. 4 shows another embodiment of a lens including a plurality of electro-active elements with refractive regions, surrounded by a dichroic crystal region, according to aspects of the invention.

FIG. 5 shows another embodiment of a lens including a plurality of electro-active elements with refractive regions according to aspects of the invention.

FIG. 6 shows another embodiment of a lens including a plurality of electro-active apertures with prismatic regions, surrounded by a dichroic crystal region, according to aspects of the invention.

FIG. 7 shows another embodiment of a lens including a plurality of electro-active apertures with prismatic regions according to aspects of the invention.

FIG. 8 shows another embodiment of a lens including a plurality of electro-active elements with diffractive regions arranged in a conformal pattern according to aspects of the invention.

FIG. 9 shows another embodiment of a lens including a plurality of electro-active elements with refractive regions arranged in a conformal pattern according to aspects of the invention.

FIG. 10 is a cross-sectional view of a lens including a diffractive region according to aspects of the invention.

FIG. 11 is a cross-sectional view of a lens including a refractive region according to aspects of the invention.

FIG. 12 is a cross-sectional view of a lens including a prismatic region according to aspects of the invention.

FIG. 13 is a cross-sectional view of a progressive electro-active lens including a diffractive region according to aspects of the invention.

FIG. 14 is a cross-sectional view of an intra-ocular electro-active lens according to aspects of the invention.

FIG. 15 is a cross-sectional view of another intra-ocular electro-active lens according to aspects of the invention.

### DETAILED DESCRIPTION OF THE INVENTION

As used herein, an electro-active element refers to a device with an optical property that is alterable by the application of electrical energy. The alterable optical property may be, for example, optical power, focal length, diffraction efficiency, depth of field, optical transmittance, tinting, opacity, refractive index, chromatic dispersion, or a combination thereof. An electro-active element may be constructed from two substrates and an electro-active material disposed between the two substrates. The substrates may be shaped and sized to ensure that the electro-active material is contained within the substrates and cannot leak out. One or more electrodes may be disposed on each surface of the substrates that is in contact with the electro-active material. The electro-active element may include a power supply operably connected to a controller. The controller may be operably connected to the electrodes by way of electrical connections to apply one or more voltages to each of the electrodes. When electrical energy is applied to the electro-active material by way of the electrodes, the electro-active material's optical property may be altered. For example, when electrical energy is applied to the electro-active material by way of the electrodes, the electro-active material's index of refraction may be altered, thereby changing the optical power of the electro-active element.

The electro-active element may be embedded within or attached to a surface of an ophthalmic lens to form an electro-active lens. Alternatively, the electro-active element may be embedded within or attached to a surface of an optic which provides substantially no optical power to form an electro-active optic. In such a case, the electro-active element may be in optical communication with an ophthalmic lens, but separated or spaced apart from or not integral with the ophthalmic lens. The ophthalmic lens may be an optical substrate or a lens.

A “lens” is any device or portion of a device that causes light to converge or diverge (i.e., a lens is capable of focusing light). A lens may be refractive or diffractive, or a combination thereof. A lens may be concave, convex, or planar on one or both surfaces. A lens may be spherical, cylindrical, prismatic, or a combination thereof. A lens may be made of optical glass, plastic, thermoplastic resins, thermoset resins, a composite of glass and resin, or a composite of different optical grade resins or plastics. It should be pointed out that within the optical industry a device can be referred to as a lens even if it has zero optical power (known as plano or no optical power). However, in this case, the lens is usually referred to as a “plano lens”. A lens may be either conventional or non-conventional. A conventional lens corrects for conventional errors of the eye including lower order aberrations such as myopia, hyperopia, presbyopia, and regular astigmatism. A non-conventional lens corrects for non-conventional errors of the eye including higher order aberrations that can be caused by ocular layer irregularities or abnormalities. The lens may be a single focus lens or a multifocal lens such as a Progressive Addition Lens or a bifocal or trifocal lens. Contrastingly, an “optic”, as used herein, has substantially no optical power and is not capable of focusing light (either by refraction or diffraction). The term “refractive error” may refer to either conventional or non-conventional errors of the eye. It should be noted that redirecting light is not correcting a refractive error of the eye. Therefore, redirecting light to a healthy portion of the retina, for example, is not correcting a refractive error of the eye.

The electro-active element may be located in the entire viewing area of the electro-active lens or optic or in just a portion thereof. The electro-active element may be located near the top, middle or bottom portion of the lens or optic. It should be noted that the electro-active element may be capable of focusing light on its own and does not need to be combined with an optical substrate or lens.

As used herein, an intraocular optic (IOO) is an optic (having substantially no optical power) that is inserted or implanted in the eye. An intraocular optic may be inserted or implanted in the anterior chamber or posterior chamber of the eye, into the stroma of the cornea (similar to a corneal inlay), or into the epithelial layer of the cornea (similar to a corneal onlay), or within any anatomical structure of the anterior chamber of the eye.

As used herein, an intraocular lens (IOL) is a lens (having optical power) that is inserted or implanted in the eye. An intraocular lens may be inserted or implanted in the anterior chamber or posterior chamber of the eye, into the capsular sac, or the stroma of the cornea (similar to a corneal inlay), or into the epithelial layer of the cornea (similar to a corneal onlay), or within any anatomical structure of the eye. An intraocular lens has one or more optical powers and may or may not also have a dynamic aperture.

As used herein, an aperture (as opposed to a micro-aperture) may refer to a first region, typically at or near the entrance pupil, that is encompassed by a second region, which may be annular. The second region may have at least one optical characteristic different than the first region. For example, the second region may have a different optical transmission, refractive index, color, or optical path length than the first region. The second region may be referred to as a peripheral region.

The invention disclosed herein relates to various embodiments of electronic ophthalmic lenses also referred to as electro-active ophthalmic lenses. Ophthalmic lens as defined herein refer to spectacle eyeglass lenses, contact lenses, intraocular lenses, or any lens that focuses, transmits, directs, and or refracts light onto the retina of the user/wearer's eye. When used as a contact lens a photo-sensor connected to an ASIC or micro controller senses the difference from a normal blink of the eye and that of a forced blink of an eye which is meant to cause the focus of the contact lens to switch from near to far or from far to near. When used as a spectacle lens a tilt switch or similar sensor connected to an ASIC or micro controller may cause the spectacle lens to change its optical power. When used as an intra-ocular lens a sensor may be used to detect a ratio of light and pupil size and may cause the intra-ocular lens to switch its optical power.

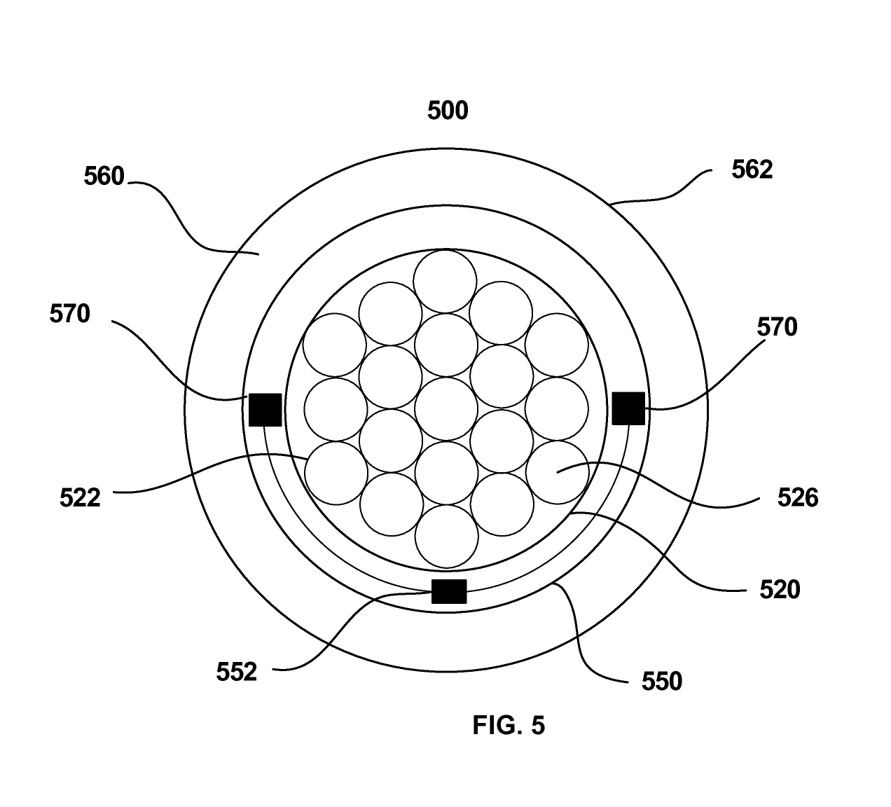

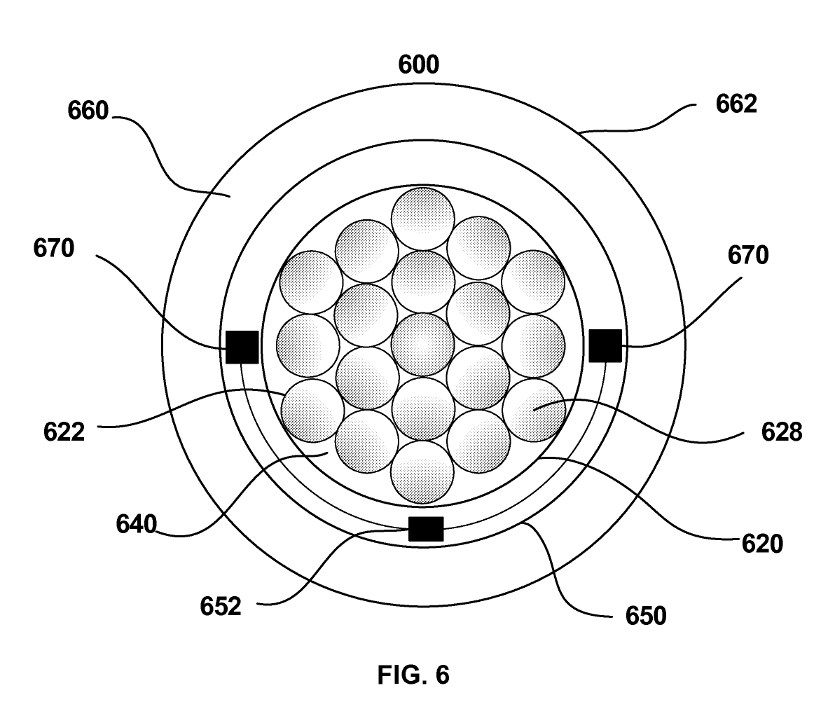

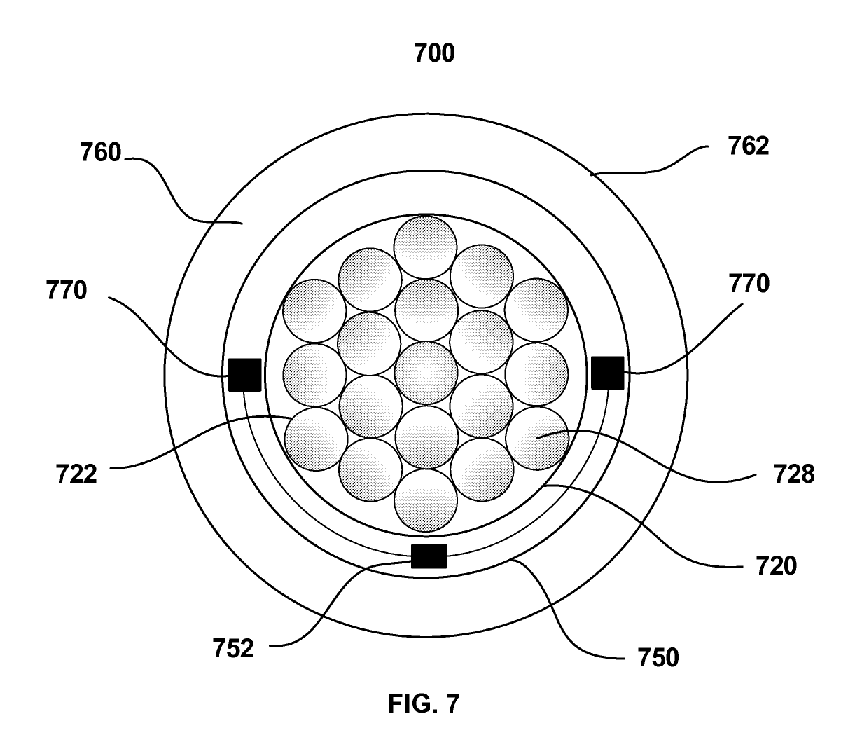

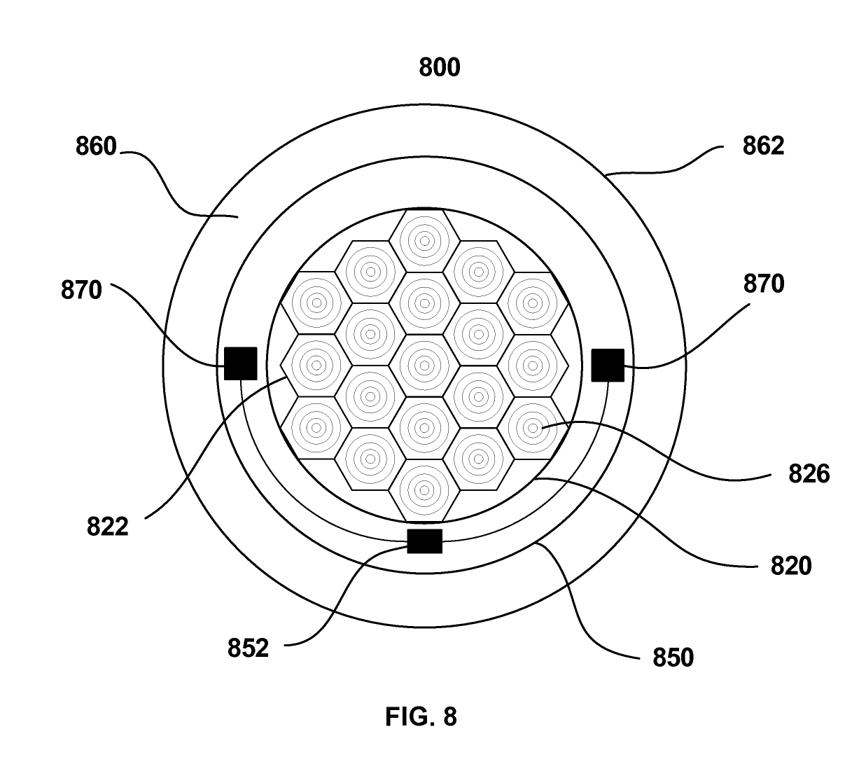

In embodiments the ophthalmic lens may include a host lens comprising one of a plurality of dynamic micro-lenses or micro-prismatic apertures. In embodiments, exemplary lenses, such as shown in Figures may 2-4, may contain a plurality of dynamic optical power regions or also called dynamic micro-lenses within an add power region. The term dynamic means the optic is capable of changeable optical power as opposed to being a fixed static optical power. The add power region is the region of the electronic lens that dynamically increases plus optical power over and beyond the distance optical power. This change can be in steps of optical power or by way of continuous optical power. Other embodiments, such as shown in FIGS. 5 and 6, may not alter optical power, but provide a plurality of dynamic appearing and disappearing micro-prismatic apertures which are also called dynamic micro-prismatic regions that increase depth of focus and redirects the light to a common point on the retina of the wearer's eye. Further embodiments, such as shown in FIGS. 7 and 8, may include a plurality of dynamic micro-lenses or dynamic prismatic apertures that are arranged in a substantially conformal pattern. For example, each dynamic micro-lens may include a hexagonal shape and be arranged in a honeycomb pattern, as shown in FIGS. 7 and 8. This allows for the plurality of dynamic micro-lenses to have a larger optical fill factor within the host lens such that a higher amount of refracted light may be focused on the retina compared to embodiments where the electro-active elements are substantially round.

It should be pointed out that, according to embodiments, given the size of each micro-lenses and its corresponding dynamic optical power, the depth of focus may be increased as the optical power is dynamically increased. This is true for the inventive ophthalmic host lens that comprises the inventive dynamic micro-lenses or micro-prismatic apertures and also for each dynamic micro-lens or micro-prismatic aperture. This is due to the fact that in most of the inventive embodiments the plurality of the dynamic micro-lenses or micro-prismatic apertures are constructed to dynamically focus or direct light to the same point on the retina of the user or wearer's eye.

However, in one inventive subset of a inventive host ophthalmic spectacle lens, the dynamic micro-lenses are designed such that some micro-lenses have the same dynamic optical power and others have different dynamic optical power, which may provide a gradient of optical power as the pupil of the eye translates horizontally left and right as well as vertically up and down across the lens surface below the fitting point of the spectacle lens. The fitting point it defined as being the point which aligns with the pupil of the eye when the wearer is looking at a far distance straight head. This gradient of optical power can mimic that of a progressive addition lens or that of a larger gradient area of usable increasing optical add power. Those skilled in the art of progressive lens optical design would readily know how to design such an optical power gradient. The inventive spectacle lens taught herein may be used, for example, for one or more of the correction of presbyopia, gaming, or entertainment.

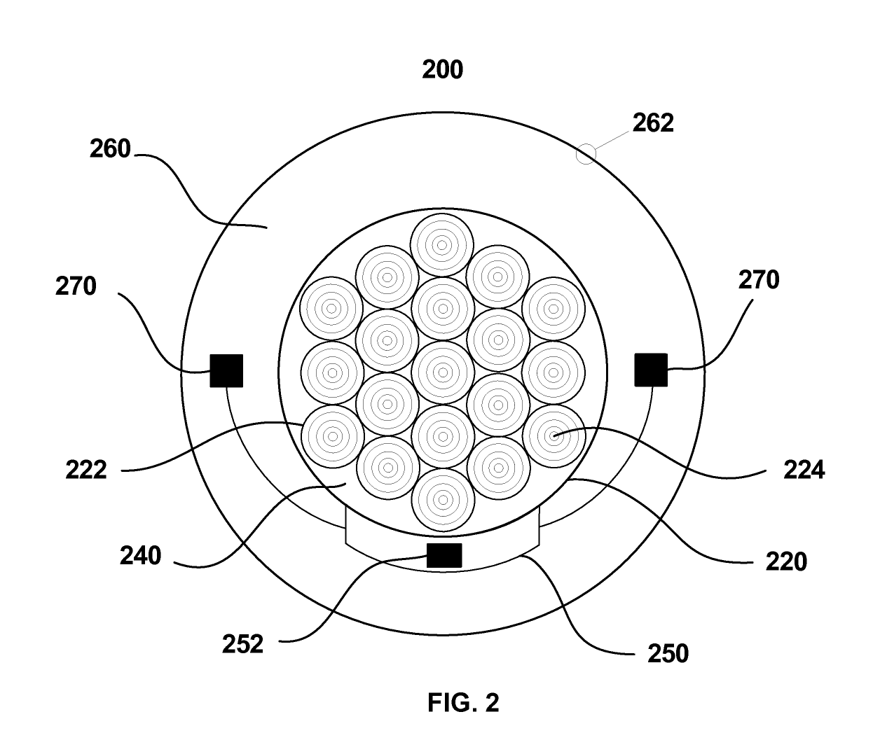

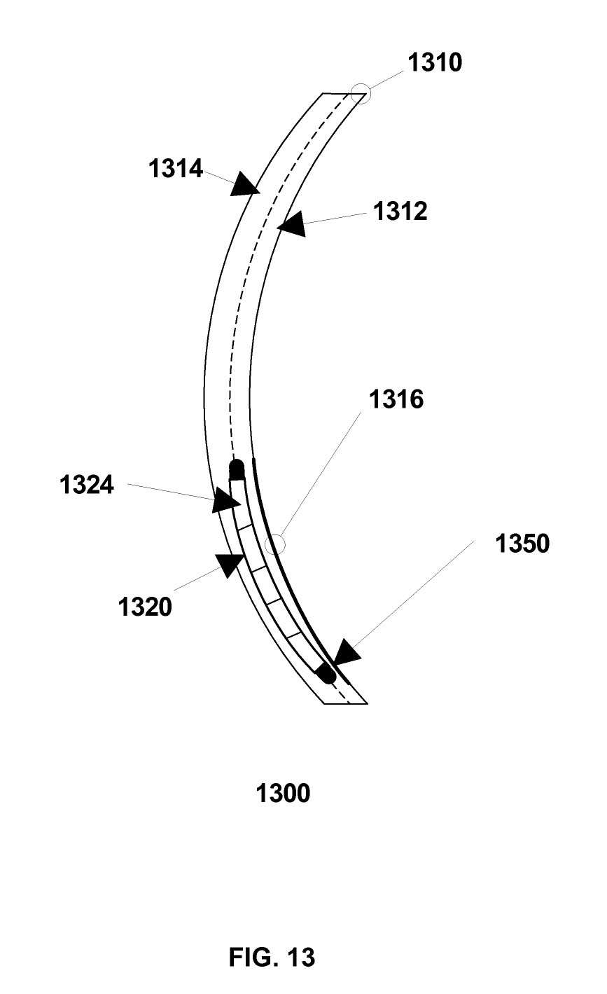

The inventive spectacle lens can also comprise dynamic micro-lenses of the same optical power in which case a low power/partial add power progressive surface may be free formed on the back side of the dynamic eyeglass lens, as shown in FIG. 13. It should be understood that, as the eye translates across the spectacle lens, the pupil of the wearer's eye acts like a stop only allowing a certain number of dynamic micro-lenses to focus light on the retina at any one time. This is true for the embodiment of when the dynamic lens is comprised of a power gradient of dynamic micro-lenses or a common power of micro-lenses, e.g. as shown in FIGS. 2-5.

In embodiments, the micro-lenses may be configured to turn on and off at the same time. In other cases, micro-lenses when individually addressed may be tuned to turn on or off at different times from one another. When such a design is used, an eye tracking system may be used to control such functions. For example, the pupil of the wearer's eye may be tracked to limit the number of micro-lenses forming an image on the retina of the wearer's or user's eye. It should also be pointed out the dynamic micro-lenses may be located along an optical designed plane such to allow for mostly one image to be formed on the retina of the eye at any one time as the pupil of the eye looks thru the ophthalmic lens.

The diameter of each micro-lens and/or prismatic aperture may be within the range of 0.5 mm to 2.0 mm and more preferably 1.0 mm to 1.60 mm. In certain cases, the dynamic micro-lenses or micro-prismatic apertures may cover the majority of the optical surface of the ophthalmic host lens that is within optical communication with the pupil of the eye of the wearer. In other embodiments, the dynamic micro-lenses or micro-prismatic apertures may cover less than the majority of the optical surface of the ophthalmic host lens that is in optical communication with the pupil of the eye of the wearer. This could be, for example, for the use of the invention with certain types of spectacle lenses and/or gaming or entertainment spectacles or eyewear.

The liquid crystal used for the inventive ophthalmic lens is, by way of example only, nematic, cholesteric. The liquid crystal can also be made to be dichroic by formulating a dichroic dye within the liquid crystal such that it will turn dark (change light absorption) when switched. In most of the inventive embodiments a single layer of cholesteric liquid crystal may be used.

The electro-active material may include a layer of liquid crystal doped with a dye material such as a dichroic dye. By doping the liquid crystal molecules with the dye material, the dye molecules align themselves with the liquid crystal molecules. The dye molecules are polar and rotate to align with an applied electrical field. The optical absorption of the dye material depends on the orientation of the individual dye molecules with respect to an incident optical wave. In a deactivated state with homogeneous (horizontal) alignment of the liquid crystal molecules, when the electric field between the electrodes is not strong enough, the dye molecules align with the alignment layers and the absorption of light through the liquid crystal is minimized or maximized, depending upon the relative orientation between the dipole moment and the direction of orientation of the dye molecule. In an activated state with homogeneous (horizontal) alignment of the liquid crystal molecules, when the electric field between the electrodes is strong enough, the dye molecules rotate and align with the orientation of the electric field, perpendicular to the alignment direction. In this orientation, the absorption of light though the liquid crystal is minimized. The opposite may be the case when a homeotropic (vertical) alignment of the liquid crystal is used such that absorption is minimized in a deactivated state and maximized in an activated state. A ferroelectric liquid crystalline material may also be used.

As described further below, embodiments of the invention may include subsets “A” and “B”, in which subset “A” includes a region of dichroic liquid crystal. However, in certain embodiments, this distinction may not apply, given, for example, the honeycomb pattern and full fill factor where there is no subset having dichroic liquid crystal. In this case only one formulation of liquid crystal may be utilized, as is with subset “B” of the above. With subset “A” the area throughout the electronic lens, with the exception of the area within the plurality of micro-lenses or micro-prismatic apertures, may be capable of having its optical transmission of light altered. For clarity this area whereby the dichoric liquid crystal is found may be around but not within, each of the dynamic micro-lenses or micro-prism apertures. The dichroic liquid crystal liquid crystal found around the micro-lens or micro-prismatic apertures may be switched such that the optical transmission of light within this region across the lens can be darkened. This is done to allow less light to be transmitted thru this region. This dichroic liquid crystal may also be capable of being switched back when desired so that the light thru this region can be increased back to the level prior to darkening.

The use of dichroic liquid crystal when switched to a darken state provides for increased contrast sensitivity for the wearer when wearing/using the inventive lenses according to embodiments. This is due to the area between and around the dynamic micro-lenses or micro-prism apertures would be darkened and therefore only the plurality of dynamic micro-lenses or micro-prismatic apertures will direct or focus only one image of light on the retina of the user or wearer's eye while the area between and around the dynamic micro-lenses or micro-apertures will not direct or focus light effectively given the darkened state. Certain embodiments may not require dichroic liquid crystal, as the fill factor, e.g. as given by a honeycomb like structure, of the micro-lenses or micro-prism apertures provides mostly a single image of light focusing on the retina of the wearer/user's eye.

Subset “B” is intended not to require having the region outside of the optical power regions and prismatic apertures altered in its transmission of light. With subset “B” there may be only one type of liquid crystal used. With inventive embodiments of subset “B” the liquid crystal may be either a dichoric liquid crystal or a non-dichroic liquid crystal.

According to embodiments of the invention, two electrodes made of transparent electrodes by way of example only, such as indium tin oxide, may be provided. One electrode may be found on the inside layer of each substrate. It should be pointed out this invention also contemplates one electrode being located on the innermost surface of one substrate and the outermost surface of the second substrate or both electrodes being located on the outermost surface of both substrates. The invention also contemplates these substrates being comprised of, by way of example only, glass, plastic or a combination of both. Subset “A” of the embodiments comprises 2 formulations of liquid crystal; dichoric liquid crystal and non-dichroic liquid crystal. Subset “A” may comprise thin walls around each micro-lens or micro-prism aperture that is only microns thick. Subset “B” may comprise only one liquid crystal formulation and in certain cases may have thin micron thick walls, whereas in other cases they may not comprise micron thick walls. The term thin micron thick walls is meant to mean within the range of 5 microns to 100 microns and most preferably 25 microns to 50 microns.

In the inventive embodiments not comprising walls around each micro-lens or micro-prism aperture one common liquid crystal formulation is provided across the plurality of micro-lenses or micro-prism apertures. The thickness of the liquid crystal layer (whether found in subset “A” or “B”) may be within the range of 1 micron and 15 microns, but preferably 3 microns to 5 microns or less.

In certain of the embodiments the micro-lenses and micro-prismatic apertures of the particular optical design are fabricated into the surface of one of two optical substrates, by way of example only, molding, diamond turning, stamping, electro-forming, thermoforming lithography, chemical or laser etching. The other substrate is a substrate having a surface curvature mostly parallel to the surface curvature of the opposing substrate.

As stated earlier with embodiments of subset “A” each of the plurality of dynamic micro-lenses and micro-prismatic apertures may comprise a peripheral thin wall which may also comprise a seal feature or lip like surface structure that is higher than the surface of the substrate within the range of 3 microns to 30 microns and is preferred to be that of a range of 3 microns to 10 microns. This peripheral wall and seal feature maintains separation of the two formulations of liquid crystal from mixing with one another.

However in certain, but not all, of subset “B” embodiments a peripheral seal feature is found around each of the micro-lenses and/or micro-prismatic apertures that comprise the lip like seal structure being of the same or very similar height within the range of 3 microns to 30 microns; by way of example only, 10 microns, In other embodiments of subset “B” there is no wall and therefore no peripheral seal feature around each dynamic micro-lens or micro-prismatic aperture. Given that subset “B” typically utilizes only one common liquid crystal formulation across the entire surface comprising dynamic micro-lenses or micro-prismatic regions a peripheral thin wall and seal feature is optional, but not mandatory.

A self contained sealed electronics module may be provided in various of the embodiments, and may comprise two substrates, two electrodes, coatings, liquid crystal, micro-lenses or micro-prismatic apertures. Once the appropriate coatings, and electrodes are deposited on the common optical surfaces of the two substrates, the two substrates may then be affixed to one another by way of example only, an adhesive and/or glass laser fusion. The substrates can be made of glass, plastic, or a combination of both. The substrates may be hermetically sealed or encased with borosilicate glass (Borofloat), by way of example only laser fusion, ionic bonding when being used for contact lenses and intra-ocular lenses after the two substrates are affixed together and have the appropriate electronics applied for making the electronic ophthalmic host lens and that of each micro-lens or micro-prismatic aperture fully functional. When utilized for contact lenses and/or intra-ocular lenses the self contained sealed electronics module (two substrates affixed to one another, liquid crystal, electrodes, coatings, electronics and hermetically sealed package) is configured to be a stand-alone optical unit that is embedded, buried, or implanted within the host ophthalmic lens. Such a stand-alone optical unit can also be called a self contained sealed electronics module.

In certain, but not all cases, this stand-alone functional optical unit can also be applied to/within a spectacle lens. However, in certain other inventive embodiments of spectacle lenses the substrates themselves are formed by way of a front lens substrate and back lens substrate. The back lens substrate can be that of a semi-finished lens blank and the front lens substrate can be that of a finished or semi-finished lens blank. In this inventive embodiments the plurality of dynamic micro-lenses or micro-prismatic apertures are formed in or on the surface of one of the host lens substrates. This surface would be that of an inner surface that would be common to the opposing parallel surface of the adjacent substrate. In this case all liquid crystal, electrodes, coatings, and electronics are sealed and buried within the spectacle lens. It should be pointed out that certain embodiments of contact lenses and intra-ocular lenses are also fabricated as just described with the spectacles when no self contained sealed electronics module is utilized. In these cases the sealing is comprised by the host ophthalmic lens material itself. By way of example, a dynamic contact lens of the invention disclosed herein can be made of a rigid plastic material surrounded by a soft hydrophilic skirt or a dynamic contact lens of the invention disclosed herein can be of a soft hydrophilic material housing a self contained sealed electronics module.

When the inventive embodiment is that of a spectacle lens the sensing is that of, by way of example only, a range finder, micro-accelerometer, tilt switch, micro-gyroscope, capacitor touch/swipe switch. Any one or all of these sensors can be built into the inventive ophthalmic host lens or that of the eyeglass frame that houses the inventive dynamic spectacle lens.

When embodiments of subset “A” are used so that the dichroic liquid crystal is not in a darkened state, the brain of the wearer/the wearer may see two images. However, due to the fact that the optical power regions and/or prismatic apertures cover the majority of the area of the electronic ophthalmic lens which is in optical communication with the pupil of the eye of the wearer the brain has a very easy time distinguishing the image being contributed by way of the optical power regions and/or prismatic apertures and suppressing the image being formed by the area not within the optical power regions and/or prismatic apertures.

When the dichroic liquid crystal is in a darkened state, the brain of the wearer/the wearer will see only one image. And the loss of light striking the retina of the wearer's eye is still of a magnitude to allow for good image quality and good vision. This is once again because the optical power regions and/or the prismatic apertures make cover the majority of the surface of the electronic ophthalmic lens in optical communication with the pupil of the wearer's eye.

For embodiments of subset “B” the eye may see two images, but, due to the fact that one image (that being the image from light focused or directed by the dynamic micro-lenses or micro prism regions) is much more pronounced than the other, the brain will readily know which image to focus on. However, given that some of the light will not be focused on the retina of the eye of the wearer there may be a loss of contrast. By increasing the fill factor, the contrast sensitivity may be improved as the vast majority of all light will form one image on the retina of the wearer or user.

An exemplary embodiment of a lens according to subset “A” of the invention is shown in FIG. 2. As shown in FIG. 2, a lens 200, such as a contact lens, may include an aperture 220 with a plurality of dynamic micro-lenses 222. The micro-lenses 222 each include a diffractive region 224. Micro-lenses 222 may be electro active and include a liquid crystal material, such as a non-dichroic material. Between the micro-lenses 222, and/or around the periphery of aperture 220, are gaps that may be filled by a liquid crystal material 240, which may be a dichroic.

Lens 200 may include a peripheral region 260 surrounding the aperture 220 and extending to a lens edge 262. The lens may further include a battery 250, such as an inductive thin-film battery, a power management system 252 and/or sensors 270, which may be, for example, photosensors. Such components may be disposed completely, or partly, within the peripheral region 260.

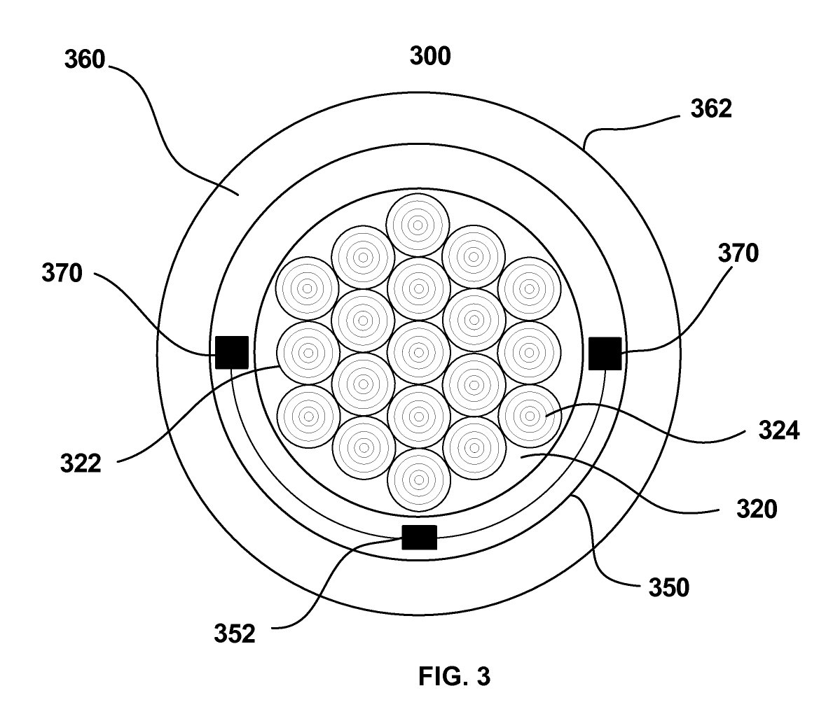

A similar exemplary embodiment of a lens according to subset “B” of the invention is shown in FIG. 3. As shown in FIG. 3, a lens 300, such as a contact lens, may include an aperture 320 with a plurality of dynamic micro-lenses 322. The micro-lenses 322 each include a diffractive region 324. Micro-lenses 322 may be electro active and include a liquid crystal material, such as a non-dichroic material.

Lens 300 may include a peripheral region 360 surrounding the aperture 320 and extending to a lens edge 362. The lens may further include a battery 350, such as an inductive thin-film battery, a power management system 352 and/or sensors 370, which may be, for example, photosensors. Such components may be disposed completely, or partly, within the peripheral region 360.

Thus, embodiments such as shown in FIGS. 2 and 3 may include a plurality of electro-active diffractive regions. Each of the plurality of the electro-active diffractive regions (dynamic micro-lenses) may provide increased optical add power when an electrical potential is applied thus changing the index of refraction of the liquid crystal to be different than that of the index of refraction of the substrate. The application of an electrical potential can be directed to each of the diffractive optical add power regions, a group of these regions, or all of the regions simultaneously. The plurality of electro-active diffractive regions as shown in FIGS. 2 and 3 are located as rings of such regions located around a single central electro-active diffractive region. The optical power of each region is in most cases of the same magnitude of optical power. The optical power of each optical power region is of the same magnitude of optical power. The optical power of these regions when activated can be within the range of +0.50 D to +4.00 D and most preferably within the range of +1.00 D to +3.00 D. When designed for spectacles or eyewear for gaming or entertainment the dynamic optical powers of each micro-lens can range from a −4.00 D to a +4.00 D

The electro-active optical region can be of a structure that is pixilated or surface relief diffractive. When pixilated it can be individually addressed, when surface relief diffractive one common set (top and bottom) of electrodes can be used. The optical power can be made to be different if desired by way of the electrode design for when pixilated or the surface relief diffractive pattern. The optical design of a diffractive optical surface capable of providing plus optical power is known in the trade. It should be pointed out that when the index of refraction of the liquid crystal found within the optical power region is equal to that of the substrate on which it is located the optical power is mostly zero and the diffractive optical power region substantially disappears.

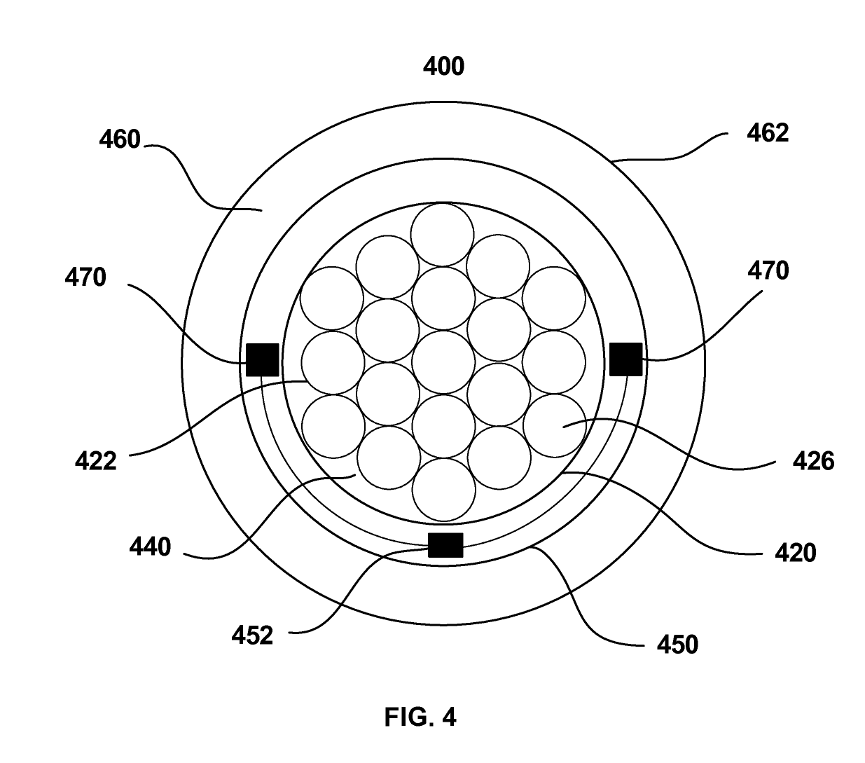

Another exemplary embodiment of a lens according to subset “A” of the invention is shown in FIG. 4. As shown in FIG. 4, a lens 400 may include an aperture 420 with a plurality of dynamic micro-lenses 422. The micro-lenses 422 each include a refractive region 426. Micro-lenses 422 may be electro active and include a liquid crystal material, such as a non-dichroic material. Between the micro-lenses 422, and/or around the periphery of aperture 420, are gaps that may be filled by a liquid crystal material 440, which may be a dichroic.

Lens 400 may include a peripheral region 460 surrounding the aperture 420 and extending to a lens edge 462. The lens may further include a capacitor 450, a power management system 452 and/or sensors 470, which may be, for example, photosensors. Such components may be disposed completely, or partly, within the peripheral region 460.

A similar embodiment of a lens according to subset “B” of the invention is shown in FIG. 5. As shown in FIG. 5, a lens 500, such as a contact lens, may include an aperture 520 with a plurality of dynamic micro-lenses 522. The micro-lenses 522 each include a refractive region 526. Micro-lenses 522 may be electro active and include a liquid crystal material, such as a non-dichroic material.

Lens 500 may include a peripheral region 560 surrounding the aperture 520 and extending to a lens edge 562. The lens may further include a capacitor 550, a power management system 552 and/or sensors 570, which may be, for example, photosensors. Such components may be disposed completely, or partly, within the peripheral region 560.

Each of the plurality of the electro-active diffractive regions (dynamic micro-lenses) shown in FIGS. 4 and 5 provides increased optical add power when an electrical potential is applied thus changing the index of refraction of the liquid crystal to be different than that of the index of refraction of the substrate. The application of an electrical potential can be directed to each of the refractive optical add power regions, a group of these regions, or all of the regions simultaneously. The plurality of electro-active refractive regions is located as rings of such regions located around a single central electro-active refractive region. The optical power of each optical power region is of the same magnitude of optical power. The optical power of each optical power region when activated can be within the range of +0.50 D to +4.00 D and most preferably within the range of +1.00 D to +3.00 D. If the electrical potential is applied such that it is not affecting all refractive optical power regions at the same time or of the same magnitude this would be accomplished by way of multiple insulated electrodes located on one or both substrates that are individually addressed.

These refractive regions can be designed, by way of example only, by way of structure of refractive curves or a Fresnel optical design. The optical design of a refractive optical surface capable of providing plus optical power is known in the trade. It should be pointed out that when the index of refraction of the liquid crystal found within the optical power region is equal to that of the substrate on which it is located the optical power is mostly zero and the refractive optical power region substantially disappears.

Another exemplary embodiment of a lens according to subset “A” of the invention is shown in FIG. 6. As shown in FIG. 6, a lens 600 may include an aperture 620 with a plurality of electro-active prismatic apertures 622. The prismatic apertures 622 each include a prismatic region 628. Prismatic apertures 622 may be electro active and include a liquid crystal material, such as a non-dichroic material. Between the prismatic apertures 622, and/or around the periphery of aperture 620, are gaps that may be filled by a liquid crystal material 640, which may be a dichroic.

Lens 600 may include a peripheral region 660 surrounding the aperture 620 and extending to a lens edge 662. The lens may further include a capacitor 650, a power management system 652 and/or sensors 670, which may be, for example, photosensors. Such components may be disposed completely, or partly, within the peripheral region 660.

A similar embodiment of a lens according to subset “B” of the invention is shown in FIG. 7. As shown in FIG. 7, a lens 700 may include an aperture 720 with a plurality of electro-active prismatic apertures 722. The electro-active prismatic apertures 722 each include a prismatic region 728. Prismatic apertures 722 may be electro active and include a liquid crystal material, such as a non-dichroic material.

Lens 700 may include a peripheral region 760 surrounding the aperture 720 and extending to a lens edge 762. The lens may further include a capacitor 750, a power management system 752 and/or sensors 770, which may be, for example, photosensors. Such components may be disposed completely, or partly, within the peripheral region 760.

The embodiments shown in FIGS. 6 and 7 include a plurality of electro-active prismatic apertures. Each of the plurality of the electro-active micro-prismatic aperture regions provides increased optical add power when an electrical potential is applied thus changing the index of refraction of the liquid crystal to be different than that of the index of refraction of the substrate. The application of an electrical potential can be directed to each of the electro-active prismatic aperture regions, a group of these regions, or all of the regions simultaneously. If this electrical potential is applied such that it is not affecting all refractive optical power regions at the same time or of the same magnitude this would be accomplished by way of multiple insulated electrodes located on one or both substrates that are individually addressed.

These prismatic apertures can be designed, by way of example only, by way of surface wedge like prism formations located within the surface of the substrate. By way of example only, such a single prism aperture can be formed so the thickness is 2 microns or less on one end and on the other end. The prism apertures are located within a series of rings around the center of the ophthalmic lens. The prismatic optical power of each series of rings is optically designed such to increase in optical prismatic “base in” power such to allow for the light being prismatically refracted to be directed to the same portion of the retina no matter which ring of prism apertures is communicating the light forming part of the image on the retina of the wearer's eye.

It should be pointed out that when the index of refraction of the liquid crystal found within the prism aperture may be equal to that of the substrate on which it is located the optical power is mostly zero and the refractive optical power region substantially disappears.

Another exemplary embodiment of a lens according to aspects of the invention is shown in FIG. 8. As shown in FIG. 8, a lens 800 may include an aperture 820 with a plurality of electro-active elements 822. In the embodiment shown in FIG. 8, the plurality of electro-active elements are substantially hexagonal and are closely formed in a honeycomb pattern, thus achieving a high fill factor. The electro-active elements 822 each include a diffractive region 824. Electro-active elements 822 may include a liquid crystal material, such as a non-dichroic material.

Lens 800 may include a peripheral region 860 surrounding the aperture 820 and extending to a lens edge 862. The lens may further include a battery 850, a power management system 852 and/or sensors 870, which may be, for example, photosensors. Such components may be disposed completely, or partly, within the peripheral region 860.

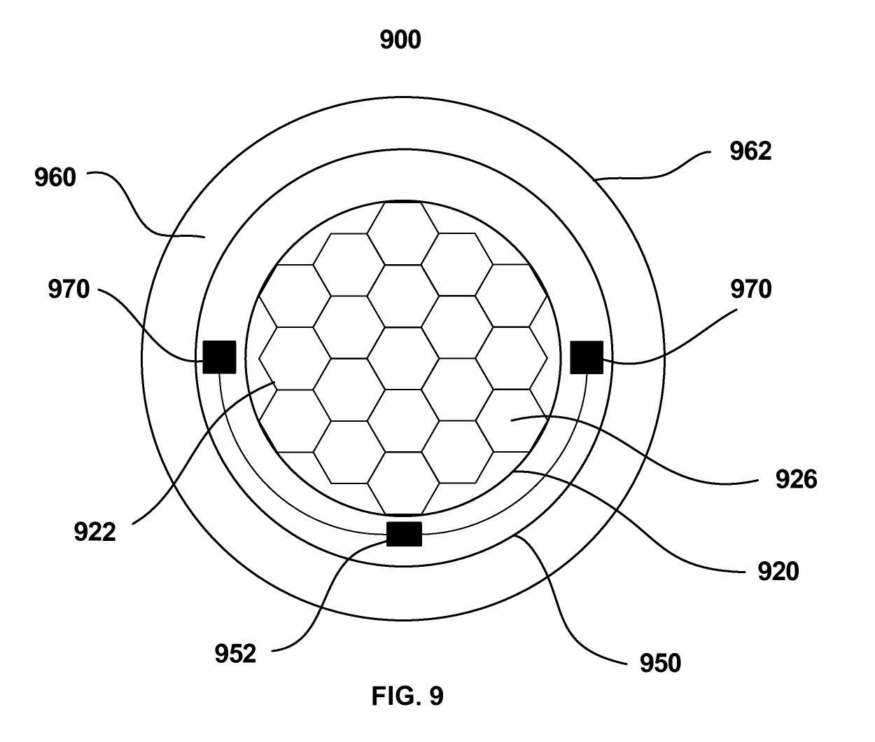

A similar embodiment of a lens according to aspects of the invention is shown in FIG. 9. As shown in FIG. 9, a lens 900 may include an aperture 920 with a plurality of electro-active elements 922. In the embodiment shown in FIG. 9, the plurality of electro-active elements are substantially hexagonal and are closely formed in a honeycomb pattern, thus achieving a high fill factor. The electro-active elements 922 each include a refractive region 926. Electro-active elements 922 may include a liquid crystal material, such as a non-dichroic material.

Lens 900 may include a peripheral region 960 surrounding the aperture 920 and extending to a lens edge 962. The lens may further include a battery 950, a power management system 952 and/or sensors 970, which may be, for example, photosensors. Such components may be disposed completely, or partly, within the peripheral region 960.

The embodiments shown in FIGS. 8 and 9 include a plurality of electro-active diffractive or refractive regions. These embodiments provide for a dynamic optical fill factor greater than those in which gaps are present between the electro-active elements. This is due to the honeycomb or hexagonal like shape of each of the micro lenses or micro-prism regions. It should be pointed out that the invention contemplates any geometrical design whereby the outer perimeter will allow for the maximum optical fill factor. Thus the outer design or each micro-lens or micro-prism aperture does not have to be that of a hexagonal, and may be instead, triangular, square, or other shapes and combinations thereof. Optical fill factor as used herein is meant to be the area of the ophthalmic lens around, between and within the micro-lenses that is capable of dynamically turning on or off optical power. In embodiments, the fill factor may be in a range of, for example, 0.8-1.0, or 0.9-1.0, or substantially 1.0.

Each of the plurality of the electro-active diffractive or refractive regions (dynamic micro-lenses) in FIGS. 8 and 9 provides increased optical add power when an electrical potential is applied thus changing the index of refraction of the liquid crystal to be different than that of the index of refraction of the substrate. The application of an electrical potential can be directed to each of the dynamic diffractive or refractive optical add power regions, a group of these regions, or all of the regions simultaneously. The plurality of electro-active diffractive or refractive regions is located within a series of rings of such regions around a single central electro-active diffractive or refractive region. The optical power of each region is in most cases of the same magnitude of optical power. The optical power of each optical power region is of the same magnitude of optical power. The optical power of these regions when activated can be within the range of +0.50 D to +4.00 D and most preferably within the range of +1.00 D to +3.00 D. However, for use with gaming and/or entertainment the optical power can be within the range of −4.00 D and +4.00 D.

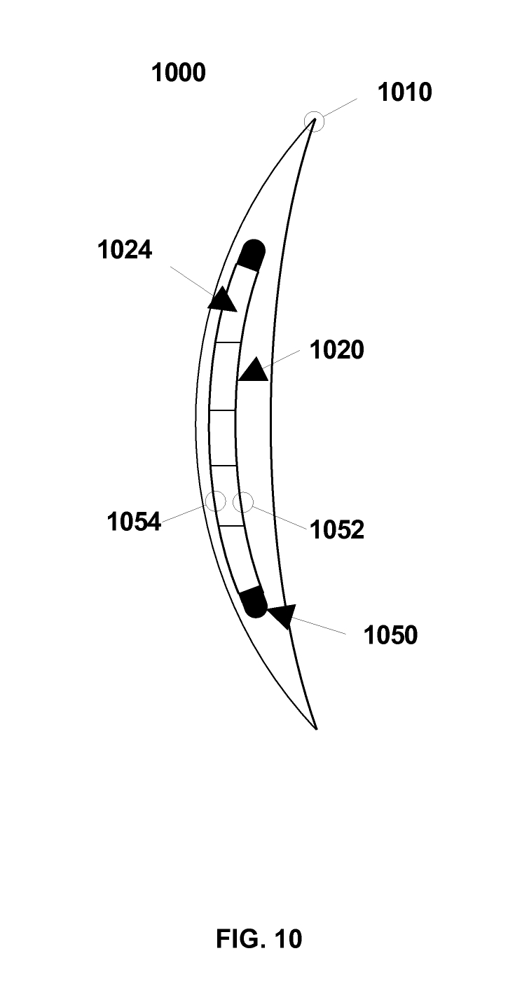

As mentioned previously, the above lens designs may be incorporated in spectacle, contact and/or intraocular lenses. Some examples of corresponding structures including such designs are shown in FIGS. 10-15.

FIG. 10 is a cross-sectional view of an exemplary lens which may include diffractive elements as previously described. As shown in FIG. 10, a lens 1000 may include a self-contained electro-active lens module 1020, similar to those discussed above, sealed an ophthalmic lens host 1010. Lens module 1020 may include a plurality of diffractive regions 1024 between first electrode 1052 and second electrode 1054. Power to the lens module 1020, and the first electrode 1052 and second electrode 1054, may be provided and/or controlled by power module 1050, which may include, for example, an inductive battery, electro-active control circuitry and power management logic.

Power module 1050 may connect to first electrode 1052 and second electrode 1054 by electrical connections and may be capable of generating an electric field between the electrodes by applying one or more voltages to each electrode. In some configurations, the module is part of the electro-active element. The module also may be located outside the electro-active element and connect to the electrodes using electrical contact points in the electro-active element. In the absence of an electric field between the electrodes, the liquid crystal molecules align in the same direction as the alignment direction. In the presence of an electric field between the electrodes, the liquid crystal molecules orient in the direction of the electric field. In an electro-active element, the electric field is perpendicular to the alignment layer. Thus, if the electric field is strong enough, the orientation of the liquid crystal molecules will be perpendicular to the alignment direction. If the electric field is not strong enough, the orientation of the liquid crystal molecules will be in a direction somewhere between the alignment direction and perpendicular to the alignment direction.

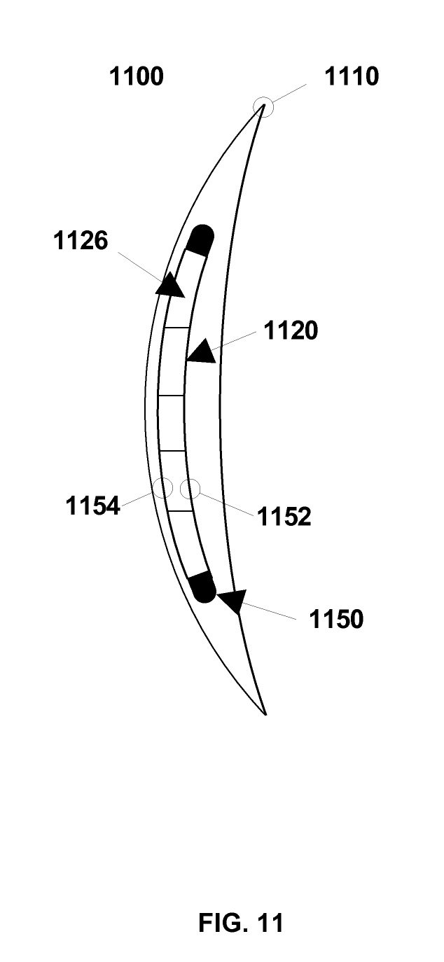

FIG. 11 is a cross-sectional view of another exemplary lens which may include refractive elements as previously described. As shown in FIG. 11, a lens 1100 may include a self-contained electro-active lens module 1120, similar to those discussed above, within an ophthalmic lens host 1110. Lens module 1120 may include a plurality of refractive regions 1126 between first electrode 1152 and second electrode 1154. Power to the lens module 1120, and the first electrode 1152 and second electrode 1154, may be provided and/or controlled by power module 1150, which may include, for example, an inductive battery, electro-active control circuitry and power management logic.

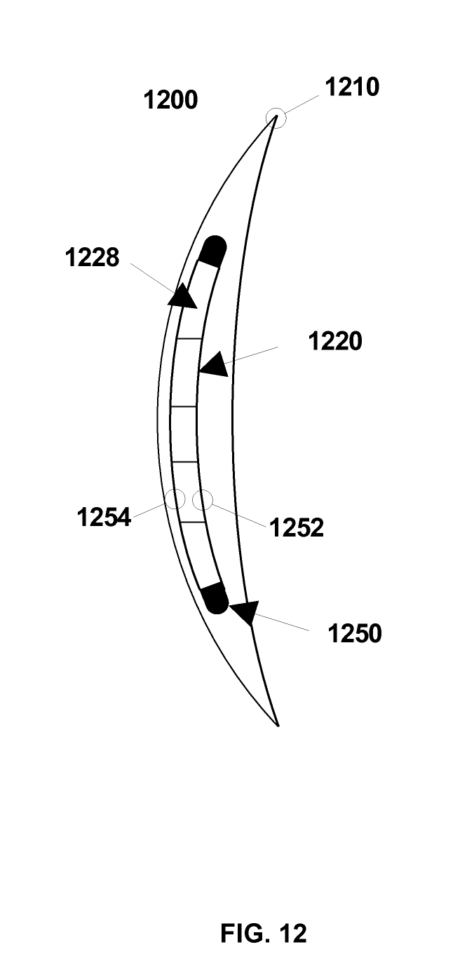

FIG. 12 is a cross-sectional view of another exemplary lens which may include prismatic elements as previously described. As shown in FIG. 12, a lens 1200 may include a self-contained electro-active lens module 1220, similar to those discussed above, within an ophthalmic lens host 1210. Lens module 1220 may include a plurality of prismatic regions 1228 between first electrode 1252 and second electrode 1254. Power to the lens module 1220, and the first electrode 1252 and second electrode 1254, may be provided and/or controlled by power module 1250, which may include, for example, an inductive battery, electro-active control circuitry and power management logic.

FIG. 13 is a cross-sectional view of an exemplary progressive lens which may include diffractive elements as previously described. As shown in FIG. 13, a lens 1300 may include a self-contained electro-active lens module 1320, similar to those discussed above, within an ophthalmic lens host 1310. Electro-active lens module 1320 may be disposed between a first substrate 1312 with a concave inner surface and a second substrate 1314 with a convex outer surface. The concave inner surface of first substrate 1312 may be, for example, a finished spectacle lens free-formed surface. Lens 1300 may also include a progressive add region, such as progressive add surface 1316. The convex outer surface of second substrate 1314 may include a spherical surface.

Lens module 1320 may include a plurality of diffractive regions 1324. Power to the lens module 1320 may be provided and/or controlled by power module 1350, which may include, for example, an inductive battery, electro-active control circuitry and power management logic.

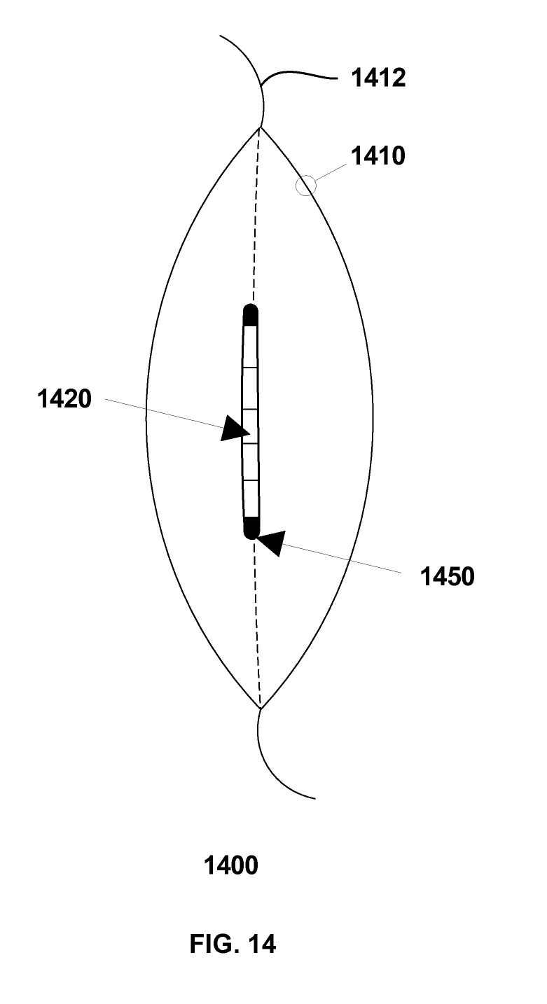

FIG. 14 is a cross-sectional view of an exemplary intra-ocular lens which may include refractive, diffractive or prismatic elements as previously described. As shown in FIG. 14, a lens 1400 may include a self-contained electro-active lens module 1420, similar to those discussed above, within an intra-ocular lens host 1410. Lens module 1420 may be configured in a substantially planar shape. In such configurations, the lens may be configured to include refractive index matching between the liquid crystal material included in the lens module 1420 and the lens host 1410. This can be matched in the activated or inactivated state. In the index matched state, the lens module 1420 may be configured to provide no additional optical power, whereas in the non-matched state, the lens module 1420 may be configured to provide additional optical power. Such configurations may be beneficial, for example, in accommodating different pupil size depending on user needs, e.g. providing no additional power from the lens module 1420 in a far-distance viewing situation where the pupil is relatively large, and providing additional optical power in the limited region of the lens module 1420 in a near-distance viewing situation where the pupil is relatively small.

Electrical power to the lens module 1420 may be provided and/or controlled by power module 1450, which may include, for example, an inductive battery, electro-active control circuitry and power management logic. Intra-ocular lens host 1410 may also include haptics 1412, or other structure suited to intra-ocular lenses.

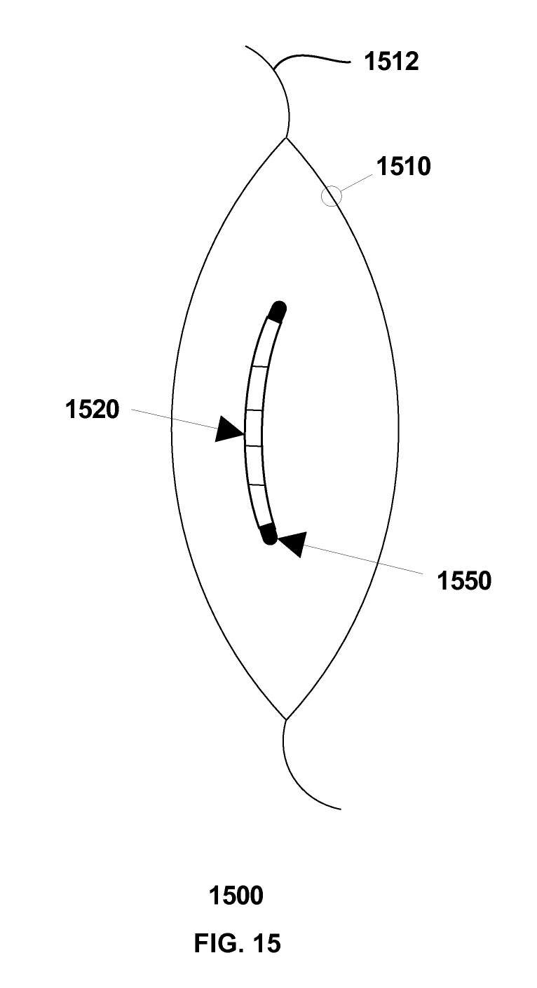

FIG. 15 is a cross-sectional view of another exemplary intra-ocular lens which may include refractive, diffractive or prismatic elements as previously described. As shown in FIG. 15, a lens 1500 may include a self-contained electro-active lens module 1520, similar to those discussed above, within an intra-ocular lens host 1510. Lens module 1520 may be configured in to include a curved profile shape. In such configurations, the lens may be configured to provide no optical power by matching a curvature of the lens module 1520 to the lens host 1510. Thus, the configuration shown in FIG. 15 can provide no additional optical power without specifically index matching the lens materials. Additional optical power may then be provided by activating the electro-active elements of lens module 1520.

Electrical power to the lens module 1520 may be provided and/or controlled by power module 1550, which may include, for example, an inductive battery, electro-active control circuitry and power management logic. Intra-ocular lens host 1510 may also include haptics 1512, or other structure suited to intra-ocular lenses.

The electro-active optical region can be of a structure that is pixilated, Fresnel or surface relief diffractive. When pixilated it can be individually addressed, when Fresnel or surface relief diffractive one common set (top and bottom) of electrodes can be used. The optical power can be made to be different if desired by way of the electrode design for when pixilated and when Fresnel or a surface relief diffractive pattern the optical design features are customized. The optical design of a refractive or diffractive optical surface capable of providing plus optical power is known in the trade. It should be pointed out that when the index of refraction of the liquid crystal found within the optical power region is equal to that of the substrate on which it is located the optical power is mostly zero and the diffractive optical power region substantially disappears.

For contact lenses each micro-lens or micro-prismatic aperture is characterized in terms of its own address relative to point of intersection of the optic axis of the eye and the anterior surface of the cornea. For intra-ocular lenses each micro-lens or micro-prismatic aperture is characterized in terms of its own address relative to point of intersection of the optic axis of the eye and principal plan of the intra-ocular lens. For spectacle lenses each micro-lens or micro-prismatic aperture is characterized in terms of its own address relative to point of intersection of the optic axis of the eye and the principal plane of the spectacle lens. Each micro-lens or micro-prismatic aperture is provided with a prismatic element that is dependent on its address, such that the transmitted image is incident on the fovea. This prismatic element may be provided by matching the anterior and posterior curvatures of the micro-lens or micro-prismatic aperture to the corresponding curvature of the overall host lens. Additionally, images produced by all the individual micro-lenses are phase matched to ensure image summation at the fovea. The depth of focus associated with each micro-lens is dependent on its “F number” and hence its aperture, since the focal lengths are all approximately equal. The image summation caused by prismatically correcting the location of each image produced by a micro-lens allows the retina to utilize a large fraction of the incident wave-front, while maintaining a large depth of focus. In summary each micro-lens is both phase matched and the appropriate prismatic element is in place to bring one largely common image to the fovea. Each micro-lens or micro-prismatic aperture whether refractive or diffractive is indexed matched with the liquid crystal when switched off within 0.0001 units of refractive index.

It should be pointed out that all measurements, dimensions, optical powers, shapes, figures, illustrations, provided herein by way of example and are not intended to be self limiting.

The liquid crystal may alter its refractive index over the visible spectrum by at least 0.1 units upon electrical activation. As used herein, the “visible spectrum” refers to light having a wavelength in the range of about 400-750 nm. A liquid crystal (LC) layer may include a guest-host mixture capable of altering the optical transmission of light upon electrical activation. As used herein, the optical transmission of a layer or device refers to the percentage of light energy that is transmitted through the layer or device and not lost to absorption or scattering. Preferably, the mixture is capable of altering the optical transmission by at least about 30%-99% upon activation. The liquid crystal layer may be pixilated as previously described, and may be electrically addressable in discrete portions of at least about 0.25 μm² without affecting the response of adjacent portions. The liquid crystal layer may be controllable by a computerized device, such as a processor and associated software, which may be capable of arbitrarily addressing multiple segments in a preprogrammed or adaptable manner. The software may be permanently embodied in a computer-readable medium, such as a special-purpose chip or a general purpose chip that has been configured for a specific use, or it may be provided by a digital signal. The software may be incorporated into a digital signal processing unit embedded into a vision correcting device.

The liquid crystalline material discussed herein may be a nematic liquid crystal, a twisted nematic liquid crystal, a super-twisted nematic liquid crystal, a cholesteric liquid crystal, a smectic bi-stable liquid crystal, or any other type of liquid crystalline material. An alignment layer is a thin film, which, by way of example only, may be less than 100 nanometers thick and constructed from a polyimide material. The thin film is applied to the surface of substrates that comes into direct contact with liquid crystalline material. Prior to assembly of the electro-active element, the thin film is typically buffed in one direction (the alignment direction) with a cloth such as velvet. When the liquid crystal molecules come in contact with the buffed polyimide layer, the liquid crystal molecules preferentially lie in the plane of the substrate and are aligned in the direction in which the polyimide layer was rubbed (i.e., parallel to the surface of the substrate). Alternatively, the alignment layer may be constructed of a photosensitive material, which when exposed to linearly polarized 1N light, yields the same result as when a buffed alignment layer is used.

To reduce power consumption, a bi-stable liquid crystalline material may be used. A bistable liquid crystalline material may switch between one of two stable states with the application of electrical power (with one state being an activated state and the other state being a deactivated state). The bi-stable liquid crystalline material remains in the one stable state until sufficient electrical power is applied to switch the bi-stable liquid crystalline material to the other stable state. Thus, electrical power is only needed to switch from one state to the other and not to remain in a state. The bi-stable liquid crystalline material may switch to a first state when +5 volts or more is applied between the electrodes and may switch to a second state when −5 volts or less is applied between the electrodes. Of course other voltages, both higher and lower, are possible.

As described above, various exemplary lenses may include embedded sensors. The sensor may be, for example, a range finder for detecting a distance to which a user is trying to focus. The sensor may be light-sensitive cell for detecting light that is ambient and/or incident to the lens or optic. The sensor may include, for example, one or more of the following devices: a photo-detector, a photovoltaic or UV sensitive photo cell, a tilt switch, a light sensor, a passive range-finding device, a time-of-flight range finding device, an eye tracker, a view detector which detects where a user may be viewing, an accelerometer, a proximity switch, a physical switch, a manual override control, a capacitive switch which switches when a user touches the nose bridge of a pair of spectacles, a pupil diameter detector, a bio-feed back device connected to an ocular muscle or nerve, or the like. The sensor may also include one or more micro electro mechanical system (MEMS) gyroscopes adapted for detecting a tilt of the user's head or encyclorotation of the user's eye.

The sensor may be operably connected to a lens controller. The sensor may detect sensory information and send a signal to the controller which triggers the activation and/or deactivation of one or more dynamic components of the lens or optic. The sensor may be a photo-detector and may be located in a peripheral region of the lens or optic and located behind the iris. This location may be useful for sensing increases and/or decreases in available light caused by the constriction and dilation of the user's pupil. The controller may have a delay feature which ensure that a change in intensity of light is not temporary (i.e., lasts for more than the delay of the delay feature). Thus, when a user blinks his or her eyes, the lens will not be changed since the delay of the delay circuit is longer than the time it takes to blink. The delay may be longer than approximately 0.0 seconds, and preferably 1.0 seconds or longer.

The sensor, by way of example only, may detect the distance to which one is focusing. The sensor may include two or more photo-detector arrays with a focusing lens placed over each array. Each focusing lens may have a focal length appropriate for a specific distance from the user's eye. For example, three photo-detector arrays may be used, the first one having a focusing lens that properly focuses for near distance, the second one having a focusing lens that properly focuses for intermediate distance, and the third one having a focusing lens that properly focuses for far distance. A sum of differences algorithm may be used to determine which array has the highest contrast ratio (and thus provides the best focus). The array with the highest contrast ratio may thus be used to determine the distance from a user to an object the user is focusing on.

Some configurations may allow for the sensor and/or controller to be overridden by a manually operated remote switch. The remote switch may send a signal by means of wireless communication, acoustic communication, vibration communication, or light communication such as, by way of example only, infrared. By way of example only, should the sensor sense a dark room, such as a restaurant having dim lighting, the controller may cause changes to the lens that impact the user's ability to perform near distance tasks, such as reading a menu. The user could remotely control the lens or optic to increase the depth of field and enhance the user's ability to read the menu. When the near distance task has completed, the user may remotely allow the sensor and controller to act automatically thereby allowing the user to see best in the dim restaurant with regard to non-near distance tasks.

The substrates described herein may be coated with materials that are biocompatible with anatomical objects in the eye. Biocompatible materials may include, for example, polyvinyldene fluoride or non-hydrogel microporous perflouroether. The substrates and the various electronics that are affixed to or embedded within the substrates may optionally be overcoated to be hermetically sealed to prevent or retard leaching. Additionally, the substrates may be designed to encapsulate the various electronics such that they are buried within the substrates.

The lenses and optics described herein may be bendable, foldable, and/or able to be rolled up for fitting during insertion through a small approximately 1 mm to 3 mm incision. A syringe-like device commonly used for implantation of IOLs having a piston may be used as an insertion tool that allows for a folded or rolled lens or optic to be placed properly where desired in either the anterior or posterior chamber of the eye.

A lens or optic that houses an electro-active element as disclosed herein can be comprised of ophthalmic materials that are well known in the art and used for IOLs, or corneal inlays. The materials can be flexible or non-flexible. For example, an IOO may be made from two approximately 100 μm layers of, for example, a polyether, a polyimide, a polyetherimide, or a polysulphone material having the appropriate electrodes, liquid crystalline material (which may be doped with a dichroic dye), optional polarizing layers, power supply, controller, sensor and other needed electronics. Each 100 μm layer is used to form a flexible envelope that sandwiches and houses the electronics and electro-active material. The total thickness of the working optic is approximately 500 μm or less. The outer diameter of is approximately 9.0 mm (not including any haptics). The IOO may be capable of being folded and inserted into the eye through a small surgical incision of approximately 2 mm or less. In some configurations, a thin layer of memory metal is utilized as part of the IOO to aid in opening the IOO to its proper shape and location after it has been inserted into the eye's anterior or posterior chamber.

An IOO or IOL including a dynamic aperture can be surgically inserted during the initial surgical procedure that inserts a conventional IOL without a dynamic aperture. Alternatively, the IOO or IOL may be surgically inserted as a follow on surgical procedure hours, days, weeks, months, or years after the initial IOL surgery.

While illustrative and presently preferred embodiments of the invention have been described in detail herein, it is to be understood that the inventive concepts may be otherwise variously embodied and employed, and that the appended claims are intended to be construed to include such variations, except as limited by the prior art.

What is claimed is:

1. An ophthalmic lens comprising:
an ophthalmic base; and
a plurality of dynamic micro-lenses, each micro-lens configured to dynamically change optical power,
wherein the ophthalmic lens is configured such that an optical power of the ophthalmic lens focuses mostly one image at one time on the retina of the eye of the wearer.

2. The ophthalmic lens of claim 1, wherein said ophthalmic lens is a spectacle lens.

3. The ophthalmic lens of claim 2, wherein the ophthalmic lens comprises a gradient of dynamic optical power.

4. The ophthalmic lens of claim 1, wherein said ophthalmic lens is a contact lens.

5. The ophthalmic lens of claim 4, wherein the contact lens is configured to switch optical power based on a lid blink.

6. The ophthalmic lens of claim 1, wherein said ophthalmic lens is an infra-ocular lens.

7. The ophthalmic lens of claim 1, wherein the dynamic micro-lenses are diffractive.

8. The ophthalmic lens of claim 1, wherein the dynamic micro-lenses are refractive.

9. The ophthalmic lens of claim 1, wherein the dynamic micro-lens is comprises a surface relief diffractive structure.

10. The ophthalmic lens of claim 1, wherein the dynamic micro-lens is comprises a pixilated structure.

11. The ophthalmic lens of claim 1, wherein the dynamic micro-lens comprises a Fresnel structure.

12. The ophthalmic lens of claim 1, wherein a diameter of the micro-lens is in a range of approximately 0.50 mm and 2.00 mm.

13. The ophthalmic lens of claim 1, wherein a diameter of the micro-lens is in a range of approximately 1.0 mm and 1.60 mm.

14. The ophthalmic lens of claim 1, wherein the ophthalmic lens is an electro-active lens.

15. The ophthalmic lens of claim 14, wherein an optical power of the electro-active lens, when activated, is in a range of approximately +1.00 D and +4.00 D.

16. The ophthalmic lens of claim 14, wherein an optical power of the electro-active lens, when activated, is in a range of approximately +1.00 D and +2.50 D.

17. The ophthalmic lens of claim 1, wherein an outer shape of each micro-lens is substantially hexagonal.

18. The ophthalmic lens of claim 1, wherein the plurality of micro-lenses are arranged within the ophthalmic base in a honeycomb pattern.

19. The ophthalmic lens of claim 1, wherein the plurality of micro-lenses are arranged within the ophthalmic base in a pattern of rings around a single micro-lens.

20. The ophthalmic lens of claim 1, wherein a shape of each micro-lens is substantially round.

21. The ophthalmic lens of claim 1, wherein each micro-lens is electronically activated.

22. The ophthalmic lens of claim 21, wherein each micro-lens comprises liquid crystal.

23. The ophthalmic lens of claim 22, wherein the liquid crystal is one of dichroic, or non-dichroic.

24. The ophthalmic lens of claim 22, wherein the liquid crystal is one of nematic, or cholesteric.

25. The ophthalmic lens of claim 1, wherein each of the each micro-lens comprises non-dichroic liquid crystal, and gaps between the micro-lenses include a dichroic liquid crystal.

26. The ophthalmic lens of claim 25, wherein the optical power of the ophthalmic lens focuses mostly one image at one time on the retina of the eye of the wearer by reducing an amount of light passing through the dichroic liquid crystal.

27. The ophthalmic lens of claim 1, wherein the optical power of the ophthalmic lens focuses mostly one image at one time on the retina of the eye of the wearer by virtue of a fill factor of an area covered by the plurality of micro-lenses.

28. An ophthalmic lens comprising:
an ophthalmic base; and
a plurality of micro-prismatic apertures wherein the ophthalmic lens is configured such that a prismatic power of said each such aperture focuses mostly one image at one time on the retina of the eye of the wearer.

29. An ophthalmic lens of claim 28, wherein said each micro-prismatic aperture is configured to dynamically change prismatic power,
and wherein the micro-apertures are configured such that a prismatic power of the micro-apertures focuses mostly one image at one time on the retina of the eye of the wearer.

30. The ophthalmic lens of claim 28, wherein a diameter of the micro-apertures is in a range of approximately 0.50 mm and 2.00 mm.

31. The ophthalmic lens of claim 28, wherein a diameter of the micro-apertures is in a range of approximately 1.0 mm and 1.60 mm.

32. The ophthalmic lens of claim 28 wherein the shape of each micro-aperture is substantially round.

33. The ophthalmic lens of claim 28 wherein the shape of each micro-aperture is substantially a hexagon.

34. The ophthalmic lens of claim 28, wherein the plurality of micro-apertures are arranged within the ophthalmic base in a honeycomb pattern.

35. An ophthalmic lens comprising:
an ophthalmic base; and
a plurality of dynamic micro-lenses, each micro-lens configured to dynamically change optical power,
wherein each of the each micro-lens comprises non-dichroic liquid crystal, and gaps between the micro-lenses include a dichroic liquid crystal.

36. The ophthalmic lens of claim 29, wherein a shape of each micro-lens is substantially round.

37. An ophthalmic lens comprising:
an ophthalmic base; and
a plurality of dynamic micro-lenses, each micro-lens configured to dynamically change optical power,
wherein the plurality of micro-lenses are shaped and arranged within the ophthalmic base in substantially conformal pattern.

38. The ophthalmic lens of claim 37, wherein an outer shape of each micro-lens is substantially hexagonal.

39. The ophthalmic lens of claim 38, wherein the plurality of micro-lenses are arranged within the ophthalmic base in a honeycomb pattern.
